(19) United States
(12) Patent Application Publication
(10) Pub. No.: US 2026/0199543 A1
(43) Pub. Date: Jul. 16, 2026

(54) DENTAL COMPOSTION HAVING INORGANIC-MODIFIED POLYMERS

(71) Applicant: Angelus Indústria de Produtos Odontológicos S/A, Londrina (BR)

(72) Inventors: William Pereira dos Santos, Londrina (BR); Bruno Henrique Fermino Goulart, Londrina (BR); Alexandre Henrique Nunes Fazan, Cambé (BR); Maíra Bendlin Calzavara, Londrina (BR)

(21) Appl. No.: 19/429,860

(22) Filed: Dec. 22, 2025

Related U.S. Application Data
(60) Provisional application No. 63/738,330, filed on Dec. 23, 2024.

(51) Int. Cl. A61L 24/00; A61L 27/02

(52) U.S. Cl. CPC A61L 24/0073; A61L 27/025; A61L 2430/12

(57) ABSTRACT

Dental compositions containing an inorganic-imprinted polymer (IIP), with high affinity of the formula components, allowing a higher conversion rate and consequently giving the cement greater biocompatibility, ensuring mechanical properties, and allowing versatile use within dentistry.

### CROSS-REFERENCE TO RELATED APPLICATIONS

This application is a U.S. nonprovisional application, which claims priority to U.S. Provisional Application No. 63/738,330 filed on Dec. 23, 2024, which is incorporated herein by reference in its entirety.

### FIELD OF THE INVENTION

The present invention is in the field of medical and dental applications. The present invention refers to a dual resin cement (photo and chemo polymerizable) comprising inorganic imprinted polymer (IIP) having ionic inorganic particles (having Ca²⁺, Mg²⁺, Zr⁴⁺, Sr²⁺, Zn²⁺, P⁻, F⁻) that is useful in dental application, and which shows high affinity of the components and higher conversion rates. The present invention also refers to the use of said composition for dental applications and to a method for inducing tissue regeneration.

### BACKGROUND OF THE INVENTION

Compositions containing functional and structural monomers, as well as photoinitiators, co-initiators, and stabilizers are widely used in dental treatments, especially dental restorations, due to their versatility, tooth-like aesthetics, adhesion capacity, low cost, practicality in application and handling, and relevant mechanical properties such as resistance to compression and flexure.

However, such compositions present some problems such as incomplete polymerization and low vinyl groups conversion rates, minimizing their effects and results. Most importantly, the presence of unconverted monomers in restorative materials (such as BisGMA, TEGDMA, and HEMA) are detrimental to biocompatibility.

Given the challenges associated with incomplete polymerization, the degree of conversion of photopolymerizable polymers is a critical parameter as it determines the efficiency of polymer network formation and directly impacts the material's stability, mechanical properties, and functional performance. Higher degrees of conversion are associated with a reduction of residual reactive double bonds and the formation of a more stable polymeric structure, resulting in improved mechanical integrity and functional behavior, as described by Lin, Y.-T.; Shie, M.-Y.; Lin, Y.-H.; Ho, C.-C.; Kao, C.-T.; and Huang, T.-H. in The Development of Light-Curable Calcium-Silicate-Containing Composites Used in Odontogenic Regeneration, Polymers, 2021, Volume 13, Article 3107.

In this context, the degree of conversion also plays a critical role in the biocompatibility of photopolymerizable dental materials. Low biocompatibility results from the pronounced cytotoxic effect, water absorption, and hydrolytic degradation, associated with a degree of incomplete polymerization, which in turn results in the leaching of unpolymerized monomers into the surrounding tissues.

Consequently, cytotoxicity and biocompatibility are key parameters for polymeric biomaterials, as residual unreacted components released after polymerization may adversely affect cell viability. Insufficient polymerization can lead to monomer elution and cellular toxicity, particularly in sensitive cell types. In vitro studies demonstrate that resin-based materials exhibit cell-type-dependent cytotoxic effects, highlighting the importance of adequate material formulation and curing to ensure biological safety, as reported by Diemer et al., In vitro cytotoxicity of different dental resin-cements on human cell lines, Journal of Materials Science: Materials in Medicine, 2021, Volume 32, Article 4.

Leaching occurs due to the high polarity of methacrylate's functional groups, since the molecular structure of water is also polar, water absorption and bond breaking of the esters present in the methacrylic functional groups takes place.

This aspect of modified resin cements undergoing hydrolysis or leaching is crucial to understanding their durability and performance in dental applications. Modified resin cements, including self-adhesive resin cements and resin-modified glass ionomer cements, are subject to several environmental and chemical factors that can influence their stability and longevity.

In this sense, and as an example, water sorption in composite resins can lead to hydrolytic degradation. When water is absorbed, it causes physical changes, such as swelling and plasticization, which facilitate hydrolysis by breaking polymer chains and cleaving functional groups through acid and oxidation reactions.

Filling in dental composites can increase resistance to hydrolytic degradation. High filling content has been associated with increased resistance to aging, as fillings can reduce the volume of polymeric matrix exposed to water and other aging media.

Furthermore, fillings can influence the pathways by which hydrolysis takes place. Water can penetrate through the interface between filling particles and the organic matrix, potentially leading to degradation of the silane coupling agent and of the fillings themselves.

The presence of fillings can affect the mechanical properties of composites during hydrolysis. Composites with fillings generally show less reduction in resistance properties compared to pure polymeric matrices, as fillings help maintain structural integrity.

The amount and type of filling can impact the hydrolytic stability of the composite. Composites with higher filling content may demonstrate better hydrolytic stability, as they reduce water sorption in the material as a whole.

These points highlight the critical role that fillings play in influencing the hydrolytic stability and overall durability of dental composites.

In order to solve these problems, in addition to improving and enhancing the effects of these compositions, additives, better quality components or adjustments in the material's polymerization are used, but in most of these options the result does not effectively improve the composition nor definitively solve the problems above indicated.

One of the possible reasons for the lack of a truly effective solution to the problems of these compositions is the lack of assessment of the chemical affinity between the components, not considering the hydrophilic and hydrophobic character of the components, as well as the fact that the composition as a whole is not considered, focusing only on a single aspect and failing to consider the synergy between the components.

The lack of affinity can compromise the properties of the cement, such as the conversion rate, incomplete formation of polymer networks and defects in the structure of the final material, such as fractures or brittleness. This can compromise the mechanical strength, durability and stability of the product, in addition to affecting the curing kinetics and the physical-chemical behavior of the system.

Regarding the conversion rate, as one of the main aspects to be considered, the assessment of the impact on the conversion degree due to the addition of inorganic fillings is essential to ensure satisfactory biocompatibility. Methods such as infrared spectroscopy (FTIR) and differential scanning calorimetry (DSC) are used to monitor the conversion of monomers into polymers and the release of unpolymerized monomers into aqueous media is analyzed to understand the potential for long-term toxicity.

It should be noted that low conversion rate leads to greater leaching of components available in the medium, with cytotoxic potential. This is because the conversion of monomers to polymers during the curing process is essential to ensure that the material structure is properly formed and stable. When the conversion rate is low, it means that a significant amount of monomers do not polymerize, remaining as free or partially reactive substances within the matrix, with a greater probability of being released and coming into contact with cells and tissues.

The incorporation of inorganic fillings influences the conversion rate in resin cements and significantly reduces the monomer conversion rate. This effect means that the presence of these inorganic fillers results in a lower degree of conversion of monomers to polymers. When modified materials are compared with unmodified commercial products, the conversion rate is reduced, achieving significantly lower monomer conversion rates, around 21-51%, compared to commercial products, which show a much higher conversion rate of 96%.

To analyze the influence of the addition of inorganic components to the organic matrix on the monomer conversion rate, it is important to understand the polymerization kinetics.

Thus, the light passing through the medium can be either absorbed or scattered. The addition of organic material causes the straight-line beams (I+λ and I−λ) to lose strength and be scattered. At the same time, part of the diffuse light is scattered backwards, in the direction of the irradiation source. The combination of these optical behaviors causes a decrease in the light intensity in the deeper layers of the material, which reduces the monomer conversion rate.

In addition, many of these unpolymerized monomers and other components, such as photoinitiators or co-initiators, can have cytotoxic properties. This means that, when leached, these compounds can cause damage to cells, inducing inflammation, cell death or even genetic alterations. This is particularly critical in materials used in direct contact with biological tissues, such as in dental treatments.

In this sense, it is necessary to verify the degree of cytotoxicity of the materials, so that they are within acceptable standards. In a dental context, acceptable cytotoxicity is low to zero levels to ensure that the materials do not cause damage to oral tissues, such as gums, teeth or mucous membranes.

Confirming the acceptable degree of cytotoxicity, in addition to keeping the material at an acceptable standard and safe for dental use, is also necessary to ensure the biocompatibility of the material. Since the higher the cytotoxicity, the lower the biocompatibility.

The biocompatibility of dental cements containing methacrylates raises concerns about cytotoxicity, since the monomers of these compounds can cause loss of cell viability in a dose-dependent manner. Research indicates that exposure to methacrylates increases oxidative stress, inflammation and alters gene regulation. Although current biocompatibility tests are largely in vitro, they do not always reflect clinical relevance, indicating the need for more standardized methods.

Clinically, most adverse reactions to methacrylate cements involve allergic responses, and sensitization and irritation tests follow standards such as ISO 10993-10 and ISO 10993-23. Adverse reactions are generally transient, but data on long-term effects are lacking. In vitro studies also show that methacrylates can cause cell cycle arrest and cell death, reinforcing the importance of careful evaluations.

Finally, the applications of conventional monomers are complicated due to post-curing shrinkage, which can reach values of up to 3% (vol) in methacrylates. The main consequences are a greater chance of filling failures and poor clinical performance due to “stress” in adhesive restorations that can lead to deformation of cusps, microleakage, decreased marginal adaptation, cracks at the interface, postoperative sensitivity and recurrent caries.

### SUMMARY OF THE INVENTION

The present invention solves the problems of low conversion rate and, consequently, low biocompatibility observed in compositions of the prior art, while ensuring improved mechanical properties and enhanced versatility for dental applications. Conventional resin cements containing directly dispersed inorganic particles often suffer from limited polymerization efficiency, heterogeneous interfaces, and reduced biological performance.

In a first and main embodiment, the present invention provides a dental composition comprising at least a micronized and densified inorganic-imprinted polymer (IIP), wherein the polymer is derived from a solid mixture of functional monomers and is imprinted with inorganic particles. The use of a polymer imprinting strategy results in the formation of a functional polymeric phase that incorporates inorganic particles within a chemically compatible polymer matrix, allowing precise control over composition and structure.

The incorporation of inorganic particles via the inorganic-imprinted polymer significantly improves the affinity between the particles and the final resin cement, since the IIP itself acts as a polymeric constituent with high compatibility with the remaining components of the resinous matrix. This improved interfacial interaction promotes homogeneous dispersion and enhanced mechanical performance. Moreover, by pre-forming the IIP under controlled polymerization conditions, the invention enables control over the polymerization process and allows the achievement of higher degrees of conversion compared to conventional resin cements in which inorganic particles are directly dispersed in the formulation, thereby contributing to superior biocompatibility and overall material performance.

In a second embodiment, the present invention provides a method for inducing tissue regeneration comprising placing the dental composition of claim 1 into the tissue to be repaired.

### BRIEF DESCRIPTION OF THE FIGURES

FIG. 1 shows the Fourier-transform infrared (FTIR) spectra for the IIP material compared with neat PMMA, highlighting the structural modifications resulting from the incorporation of akermanite. The analysis reveals reduced intensities, peak shifts, and new absorption bands, indicating higher monomer conversion, fewer residual carbonyl groups, and interactions between the polymer matrix and inorganic species. These findings confirm that the IIP represents a molecularly modified system rather than a simple physical mixture.

FIG. 2 shows the synthesized materials IIP analyzed by scanning electron microscopy (SEM) at 3000× magnification. It is possible to observe in the samples that the akermanite bioceramic particles were imprinted on the PMMA polymer matrix, indicating that the polymer was formed using inorganic imprints.

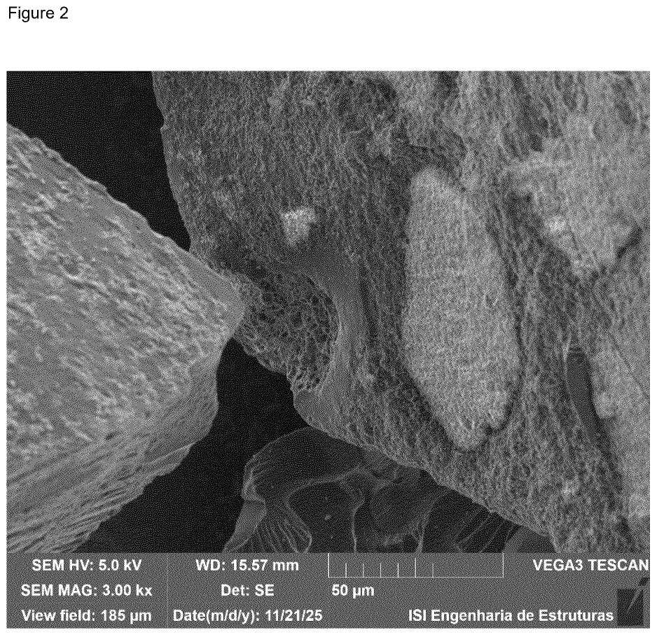

FIG. 3 shows the evaluation performed using scanning electron microscopy (SEM) under 1000× magnification (FIG. 3A) coupled with energy-dispersive X-ray spectroscopy (EDS) (FIG. 3B) on PMMA and IIP samples in milled powder form. FIG. 3B micrograph shows carbon and oxygen signals. FIG. 3 C shows only carbon signals. FIG. 3D show only oxygen signals.

FIG. 4 shows the evaluation performed using scanning electron microscopy (SEM) under 250× magnification (FIG. 4A) coupled with energy-dispersive X-ray spectroscopy (EDS) (FIG. 4 B) on PMMA and IIP samples in solid form. The FIG. 4 micrographs highlight morphological differences, and EDS analysis of the IIP reveals the presence of silicon (FIG. 4C), magnesium (FIG. 4D), and calcium (FIG. 4G) from akermanite, along with carbon (FIG. 4E) and oxygen (FIG. 4F) from the PMMA matrix.

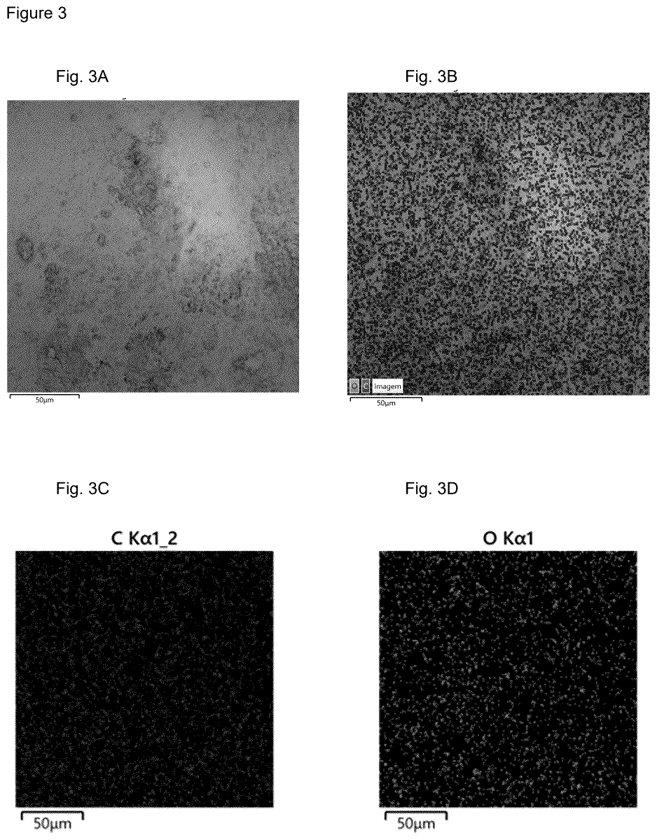

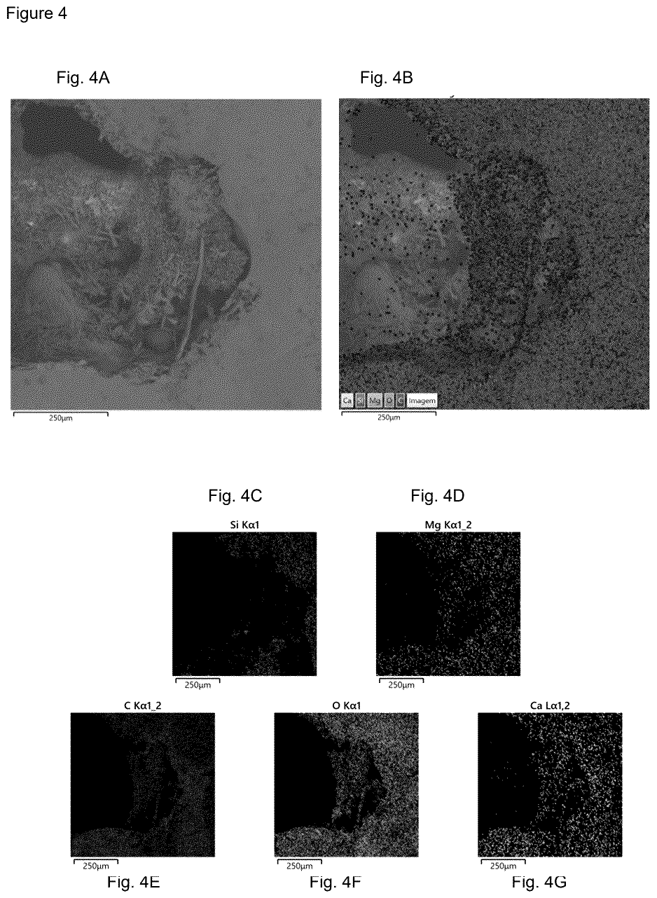

FIGS. 3 and 4 confirm the incorporation of inorganic elements and the macroscopic chemical interaction between the ceramic phase and the polymer. Further, EDS analysis of the IIP at both magnifications reveals the elemental presence of silicon, magnesium, and calcium from akermanite, along with carbon and oxygen from the PMMA matrix. In contrast, PMMA powder shows only carbon and oxygen signals (and traces of sodium, likely due to minor sample contamination). At 1000× magnification, molecular-level interactions between the inorganic phase and the polymer become more apparent, supporting the imprinting of ceramic particulates onto the polymeric matrix.

### DETAILED DESCRIPTION OF THE INVENTION

Although the present invention may be susceptible to different embodiments, the drawings and the following detailed discussion show a preferred embodiment with the understanding that the present embodiment should be considered an exemplification of the principles of the invention and is not intended to limit the present invention to what has been illustrated and described in this application.

It should also be understood that the terminology used herein is for the purpose of describing particular embodiments only and is not intended to be limiting, since the scope of the present invention will be limited only by the appended claims.

Unless otherwise defined, all technical and scientific terms used herein have the same meaning as commonly understood by one of ordinary skill in the art to which this invention pertains. Although any methods and materials similar or equivalent to those described herein may be used in the practice or testing of the present invention, some potential and exemplary methods and materials can now be described.

Any and all publications referenced herein are hereby incorporated by reference to disclose and describe the methods and/or materials in connection with which the publications are cited. It is understood that this disclosure supersedes any disclosure of an incorporated publication to the extent that there is a contradiction.

In a first and main embodiment, the present invention provides a dental composition comprising at least a micronized and densified inorganic-imprinted polymer (IIP), wherein the polymer is derived from a solid mixture of functional monomers and is imprinted with inorganic particles.

The development of resin cement containing inorganic particles aims to provide greater affinity between the matrix and the monomers within its composition, promoting less interference in the conversion rate.

Once the inorganic particles are incorporated into the resin cement, there will be greater biocompatibility, without compromising mechanical resistance, reactivity and aesthetic compatibility.

One of the strategies for this is the development of inorganic-imprinted polymers (IIP), which allow the immobilization of inorganic particles. This improvement in immobilization aims to obtain greater control of the polymerization process, with high polymer conversion rates and high chemical stability, and consequently are more resistant to degradation and less cytotoxic.

IIPs are materials designed to present a specific structure that allows ion exchange, and in a rechargeable manner (self-regulation due to the concentration of ions in the physiological environment), during controlled releases of ions. This technology uses a mold (or “imprint”) of inorganic particles, particularly, silicates, that form cavities or active sites in the polymer matrix, which will be ready to carry out ion exchanges with moisture.

IIP allows the optimization of conversion during its manufacturing process, since it is possible to modulate its parameters, notably: (a) homogenization (promotes greater stabilization of the dispersion of monomers and inorganic particles), (b) temperature (allows the ideal temperature to be reached) and (c) time (is measured with greater precision), and all of these parameters directly influence the quality of the polymerization.

It should be emphasized that these advantages would not be achieved by the use of bioceramic particles directly added to the cement.

The conversion rate will also be improved by this same process, since the parameters described above can be modulated more easily, seeking ideal rates.

Another advantage is the wide possibility of choosing the components, as it allows for greater affinity and, consequently, greater chemical stability.

The main functionality of IIPs is their ability to exchange ions with a solution, maintaining them stably within their structure, and releasing them in a controlled manner when necessary. IIPs combine the stability and durability of silicates with the specificity of interaction provided by imprinting, resulting in highly selective and efficient materials for ion manipulation.

In addition to these advantages, there is no evidence of the use of IIPs technology as proposed in the present application for dentistry. The improvement in the immobilization and use take place before photopolymerization, and is one of the main features that distinguish the present invention from the technology already disclosed in the prior art.

Polymeric materials incorporating inorganic particles and molecular or ion-imprinted structures are traditionally associated with sensing and separation technologies, as they enable selective recognition sites within a stable polymeric matrix. The incorporation of inorganic components and controlled crosslinking during polymerization contributes to improved structural stability, selectivity, and binding efficiency, allowing the formation of specific cavities complementary to target species. Such imprinted polymer particles have been demonstrated to function effectively as selective solid-phase extractors and recognition elements, as described in Ramakrishnan et al., Synthesis of Ion Imprinted Polymer Particles, U.S. Pat. No. 6,960,645 B2, granted Nov. 1, 2005.

Additionally, when compared to the cement already in use in the prior art, the use of IIPs improves its interaction with the components of the formula, in order to facilitate hydrophobic interactions and contribute to the mechanical properties of the product.

In contrast to the prior art, the present invention provides a polymeric matrix incorporating inorganic particles that are previously prepared and integrated into the polymer system in a controlled manner, thereby allowing improved control over particle distribution, interfacial interactions, and overall functional performance of the material. By comparison, US 2008/0318190 A1 describes the direct addition of a ceramic matrix into the resin cement composition, in which the inorganic phase is incorporated as a conventional filler without any prior treatment, surface modification, or controlled integration into a functional polymeric matrix, thus limiting control over chemical interactions and the resulting material performance.

Contrary to the present invention, which provides a polymeric matrix incorporating inorganic particles that are previously prepared and integrated into the polymer system in a controlled manner, EP 3 708 140 A1 discloses bioactive compositions in which ceramic or inorganic components are added directly to the resin-based cement formulation. In the cited patent, the inorganic phase functions as a conventional bioactive or hydraulic filler that is incorporated without prior surface treatment, functional modification, or controlled integration into a pre-defined polymeric matrix. As a result, the ceramic component is dispersed directly within the cement composition, whereas the present invention enables improved control over particle-polymer interactions, spatial distribution, and functional performance by employing a polymeric matrix specifically designed to host and interact with the inorganic particles.

In a preferred embodiment the synthesis of the IIP requires reagents well known in the prior art that works as functional monomers, inorganic particles, crosslinker, initiator, and inhibitor.

Functional monomers are units that form the IIP's polymeric structure, which grows around the inorganic particles.

In a preferred embodiment, the polymer is derived from the group of methacrylate monomers modified with silicates and ionic salts.

In a further preferred embodiment, the polymer derived from the group of methacrylate monomers selected from the group of methacrylic acid derivatives, or phosphocholine derivatives, or ethoxylated monomers or any other monomer capable of forming polymeric chains.

Suitable functional monomers can be selected from compounds derived from methacrylic acid (HEMA, EMA, MAA, UDMA, among other substances of the same group), methacrylic compounds containing a phosphocholine group (such as 2-Methacryloyloxyethyl Phosphorylcholine—MPC), ethoxylated monomers or any other compound capable of forming polymeric chains. Particularly, preferred functional monomers can be selected from Methyl methacrylate (MMA) derivatives, Bis-GMA derivatives, Bis-EMA derivatives, Urethane derivatives methacrylate (UDMA), Hydroxymethyl methacrylate (HEMA) derivatives, and Phosphorylcholine derivatives (MPC).

The inorganic particles suitable for the present invention are capable of exchanging ions in contact with water.

In a preferred embodiment, the inorganic particles are silicates and/or ionic salts. Non-limiting examples can be selected form the group consisting of salts containing ions (Ca, Mg, Sr, Zn, among others), silicates containing ions (Ca, Mg, Sr, Zn, among others), aluminates (Ca, Mg, Sr, Zn, among others), titanates (Ca, Mg, Sr, Zn, among others), and phosphosilicates (Ca, Mg, Sr, Zn, among others).

In a preferred embodiment, the inorganic particles are silicates.

By forming crosslinks between monomers, crosslinkers connect the polymer chains to each other and stabilize the IIP's structure by improving its mechanical properties, such as mechanical strength and rigidity. Compounds useful as crosslinks include methacrylic acid derivatives, EGDMA, 2-methacryloylethyl methacrylate (MAEMA) and Trimethylolpropane trimethacrylate (TRIM).

The initiators, under heat, generate free radicals that react with the monomers and crosslinkers to initiate polymerization. Dicumyl peroxide, tert-butyl peroxide, benzoyl peroxide, tert-butyl peroxyacetate, tert-butyl peroxybenzoate, AIBN (azobisisobutyronitrile) are non-limiting examples of compounds that can be used as initiators. Particularly, preferred initiators suitable for the invention are camphor quinone and benzoyl peroxide.

In addition, co-initiatior, such as a tertiary amine, can also be used to aid polymerization.

On the other hand, the inhibitors control the reaction kinetics, slowing down the polymerization and promoting greater control of the binding sites. 2,6-di-tert-butyl-p-cresol, butylhydroxytoluene derivatives and hydroquinones are examples of inhibitors suitable for the present invention. Particularly, preferred inhibitors are BHT, antioxidant.

To obtain the IIP of the invention, the inorganic particles and the functional monomer are mixed in the appropriate stoichiometric proportion in the presence of pore-dispersing solvents that facilitate the dispersion of the inorganic matrix particles in the polymer. The mixing is carried out under continuous homogenization, at temperature range between 5° and 80° C. to increase the miscibility between monomers and inorganic particles and, preferably, in an inert atmosphere, that is, in the absence of oxygen. The inhibitor can be added at this stage and an initial aggregate formed from intermolecular bonds between the functional monomer and the inorganic particles is obtained.

The crosslinker and initiator are added to the initial aggregate, the initiator generated free radical that reacts with the functional monomers, starting the polymerization of the functional monomers and forming the IIP chains. The polymer chains are gradually linked together by the crosslinkers. This process may also be carried out in an inert atmosphere.

Once all the reagents have been added, the mixture is poured into a mold and subjected to heat treatment for polymerization for an estimated time of 12 to 72 hours. During this time, the reaction continues with the formation of chains and crosslinks, involving the particles of the inorganic particles matrix in the polymer.

After the thermal polymerization, the formed product is cooled to room temperature and ground, becoming a fine powder. Grinding is followed by sieving to select the desired particle size range. The micronized IIP is stored in hermetically sealed containers protected from moisture. The micronized IIP is stored at humidity levels below 50%.

While a preferred embodiment of the present invention is shown and described herein, one skilled in the art will appreciate in the appended claims that many modifications and variations of the present invention are possible in light of the above teachings. It is therefore, to be understood that, within the scope of the appended claims, the invention may be practiced otherwise than as specifically described.

In a preferred embodiment, the dental compositions of the present invention further comprise suitable carrier or excipients.

Particularly, the dental compositions of the present invention may be formulated with any photo or chemo polymerized cement of the prior art.

The use of the dental composition having the micronized IIP will provide, after photopolymerization, ionic exchanges with the medium. Examples of reaction mechanism of ion exchange are as follows:

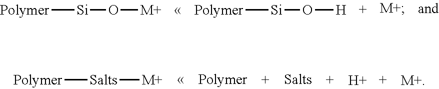

The constant concentration of multiple metal ions in the medium allows enzymatic changes that influence and stimulate tissue formation, promoting repair and regeneration of the affected area, giving the dental composition bioactive properties.

Additionally, due to the combined inorganic and polymeric characteristics of the composition, and particularly to the high specificity provided by the imprinted polymer surface containing inorganic particles containing specific ions, selective ion exchange processes are enabled. This ion exchange is reversible and rechargeable, since the polymeric matrix allows continuous ion intercambiability with the surrounding medium, occurring both under hypo-saturated and hyper-saturated ionic conditions, thereby enabling controlled release and reuptake of ions according to the ionic gradient of the medium. This dynamic behavior maintains long-term bioactivity and a sustained therapeutic effect.

In a further embodiment, the present invention provides the use of the dental compositions of the present invention for dental and medical applications.

In a further embodiment of the invention, a method for inducing tissue regeneration comprising the placing the dental compositions of the present invention into the tissue to be repaired is provided.

While a preferred embodiment of the present invention is shown and described herein, one skilled in the art will appreciate in the appended claims that many modifications and variations of the present invention are possible in light of the above teachings. It is therefore, to be understood that, within the scope of the appended claims, the invention may be practiced otherwise than as specifically described.

The present invention further contemplates the use of the material referred to herein as IIP in dental compositions comprising polymeric components. Such dental compositions include, without limitation, epoxy- or methacrylate-based sealants, obturation cones or points, thermoplastic obturation systems, temporary cements, temporary restorative materials, resin fiber posts, and similar polymer-containing formulations. In such materials, the polymers conventionally employed may be wholly or partially replaced or combined with the polymeric matrix of IIP so as to confer the technical advantages described herein, including improved curing behavior, reduced cytotoxicity, and enhanced mechanical strength. These advantages arise, at least in part, from the inorganic constituents embedded within the IIP.

### Example

IIP composition of the present invention (Table 1) are prepared by mixing the liquid carrier component and the complexing resin with a mechanical stirrer and then adding the bioceramic component:

TABLE 1
Examples of the IIP Compositions.

| Sample | Bioceramic | Monomer 1 | Monomer 2 | Monomer 3 | Initiator |
|---|---|---|---|---|---|
| TP1 | Akermanite | Methyl | Triethylene glycol | Urethane | Phenylbis (2,4,6- |
| | (Ca₂MgSi₂O₇) | Methacrylate | dimethacrylate | dimethacrylate | trimethylbenzoyl) |
| | 20% | (MMA) | (TEGDMA) | (UDMA) | phosphine oxide |
| | | 47% | 24% | 8% | (BAPO) ≤1% |
| TP2 | Baghdadite | Methyl | Triethylene glycol | Ethylene Methyl | Phenylbis (2,4,6- |
| | (Ca₂ZrSi₂O₇) | Methacrylate | dimethacrylate | Acrylate | trimethylbenzoyl) |
| | 20% | (MMA) | (TEGDMA) | copolymer (EMA) | phosphine oxide |
| | | 50% | 25% | 4% | (BAPO) ≤1% |
| TP3 | Akermanite | Bisphenol A | Triethylene glycol | Urethane | Phenylbis (2,4,6- |
| | (Ca₂MgSi₂O₇) | glycidyl | dimethacrylate | dimethacrylate | trimethylbenzoyl) |
| | 40% | methacrylate | (TEGDMA) | (UDMA) | phosphine oxide |
| | | (Bis-GMA) | 13% | 8% | (BAPO) ≤1% |
| | | 38% | | | |
| TP4 | Baghdadite | Bisphenol A | Triethylene glycol | Ethylene Methyl | Phenylbis (2,4,6- |
| | (Ca₂ZrSi₂O₇) | glycidyl | dimethacrylate | Acrylate | trimethylbenzoyl) |
| | 40% | methacrylate | (TEGDMA) | copolymer (EMA) | phosphine oxide |
| | | (Bis-GMA) | 17% | 4% | (BAPO) ≤1% |
| | | 38% | | | |
| TP5 | Akermanite | Methyl | 2- | Urethane | Benzoil peroxide |
| | (Ca₂MgSi₂O₇) | Methacrylate | methacryloyloxyethyl | dimethacrylate | (BPO) ≤1% |
| | 20% | (MMA) | phosphorylcholine | (UDMA) | |
| | | 68% | (MPC) | 8% | |
| | | | 3% | | |
| TP6 | Baghdadite | Methyl | 2- | Ethylene Methyl | Benzoil peroxide |
| | (Ca₂ZrSi₂O₇) | Methacrylate | methacryloyloxyethyl | Acrylate | (BPO) ≤1% |
| | 20% | (MMA) | phosphorylcholine | copolymer (EMA) | |
| | | 72% | (MPC) | 4% | |
| | | | 3% | | |

pH, ion release, anti-inflammatory, bioactivity and antimicrobial assays were conducted with the compositions prepared in the Example.

### pH Assay

For the pH assay a small amount of the compositions were added into Eppendorf flasks and carried out in triplicate. Then, 1 mL of distilled and deionized water was added. The pH assay was carried out with a 900 μL aliquot of the supernatant retrieved after centrifugation at 1,400 RPM for 3 minutes. After each assay, in 24 h (1 day), 3, 5, 10, 20 and 30 days, 900 μL of distilled and deionized water was added into each sample to enable the ion exchange.

### Metal Ion Release Assay

For the metal ion release assay, a small amount of the compositions were added into Eppendorf flasks and carried out in triplicate. Then, 1.5 mL of distilled and deionized water was added. The metal ion release assay was carried out with a 1,000 μL aliquot of the supernatant retrieved after centrifugation at 10,000 rpm for 3 minutes. The 1,000 μL aliquot was then transferred to a 5 mL beaker and diluted to 2 mL with distilled water. The assay was performed using the pH meter with a properly calibrated calcium electrode. After each assay, in 24 h (1 day), 3, 5, 10, 20 and 30 days, 1,000 μL of distilled and deionized water was added into each sample to enable the ion exchange.

Table 2, below, shows the results obtained in the pH and metal ions release assays. The results show that the compositions presented pH≈10 that is sufficient for antimicrobial activity and constant release of metal ions during the tested period of 30 days.

TABLE 2
Results of the physical-chemical assays of the bioceramic compositions.

| | Ionic concentration (cumulative) (ppm) | | | | | | | | | | | | pH | | | | | |
|---|---|---|---|---|---|---|---|---|---|---|---|---|---|---|---|---|---|---|
| Days | TP 1 | | TP 2 | | TP 3 | | TP 4 | | TP 5 | | TP 6 | | TP 1 | TP 2 | TP 3 | TP 4 | TP 5 | TP 6 |
| 1 | Ca | (154) | Ca | (50) | Ca | (202) | Ca | (63) | Ca | (218) | Ca | (69) | 8.00 | 7.60 | 8.20 | 7.80 | 8.20 | 7.80 |
| | Mg | (46) | Zr | (56) | Mg | (60) | Zr | (70) | Mg | (66) | Zr | (78) | | | | | | |
| | Si | (108) | Si | (35) | Si | (140) | Si | (44) | Si | (152) | Si | (48) | | | | | | |
| 3 | Ca | (373) | Ca | (122) | Ca | (490) | Ca | (152) | Ca | (528) | Ca | (168) | 8.20 | 7.80 | 8.40 | 7.90 | 8.40 | 8.00 |
| | Mg | (114) | Zr | (138) | Mg | (147) | Zr | (172) | Mg | (159) | Zr | (188) | | | | | | |
| | Si | (262) | Si | (86) | Si | (341) | Si | (106) | Si | (368) | Si | (116) | | | | | | |
| 5 | Ca | (511) | Ca | (167) | Ca | (672) | Ca | (210) | Ca | (724) | Ca | (230) | 8.20 | 7.90 | 8.40 | 8.00 | 8.60 | 8.00 |
| | Mg | (156) | Zr | (190) | Mg | (202) | Zr | (236) | Mg | (218) | Zr | (258) | | | | | | |
| | Si | (360) | Si | (117) | Si | (468) | Si | (146) | Si | (504) | Si | (160) | | | | | | |
| 10 | Ca | (673) | Ca | (216) | Ca | (880) | Ca | (271) | Ca | (949) | Ca | (298) | 8.40 | 8.00 | 8.60 | 8.00 | 8.70 | 8.20 |
| | Mg | (204) | Zr | (246) | Mg | (267) | Zr | (309) | Mg | (288) | Zr | (340) | | | | | | |
| | Si | (474) | Si | (152) | Si | (616) | Si | (190) | Si | (664) | Si | (210) | | | | | | |
| 20 | Ca | (752) | Ca | (241) | Ca | (984) | Ca | (303) | Ca | (1061) | Ca | (333) | 8.45 | 8.05 | 8.65 | 8.05 | 8.75 | 8.25 |
| | Mg | (228) | Zr | (275) | Mg | (298) | Zr | (345) | Mg | (322) | Zr | (380) | | | | | | |
| | Si | (530) | Si | (170) | Si | (688) | Si | (212) | Si | (742) | Si | (235) | | | | | | |
| 30 | Ca | (776) | Ca | (249) | Ca | (1015) | Ca | (312) | Ca | (1094) | Ca | (344) | 8.50 | 8.10 | 8.70 | 8.10 | 8.80 | 8.30 |
| | Mg | (235) | Zr | (284) | Mg | (308) | Zr | (356) | Mg | (332) | Zr | (392) | | | | | | |
| | Si | (546) | Si | (175) | Si | (710) | Si | (219) | Si | (766) | Si | (242) | | | | | | |

By way of previous examples, an illustrative IIP composition was prepared according to the following formulation (percentages by weight):
70% to 80% of a selected monomer, in the present example methacrylic acid;
20% to 30% of an inorganic material, exemplified herein by akermanite, a silicate-based bioceramic belonging to the calcium-magnesium silicate family and having the chemical formula Ca₂MgSi₂O₇; and
up to 1% of a polymerization initiator, such as benzoyl peroxide.

Following synthesis and comminution, the resulting material comprises inorganic akermanite particles molecularly imprinted within a polymethyl methacrylate (PMMA) polymeric matrix. Comparative evaluation of IIP relative to the pure polymer (PMMA) demonstrates differences in physicochemical properties that substantiate the technical improvements disclosed herein.

One evaluation considered is pH analysis in aqueous dispersion. The presence of inorganic constituents in IIP tends to raise the pH of the surrounding medium. In the representative test performed, approximately 1 g of each sample (PMMA and IIP) was dispersed in 4 mL of water. Although PMMA is effectively insoluble, immersion yielded a pH of approximately 4.5. IIP, which is likewise insoluble, exhibited an elevated pH of approximately 6.0, attributed to ionic release from the inorganic particles imprinted within the polymeric matrix, specifically calcium and magnesium ions.

It is well established that alkaline pH conditions promote antimicrobial activity, stimulate mineralization and tissue repair, neutralize inflammatory environments, prevent dentin-matrix degradation, and improve biocompatibility. These beneficial effects are not characteristic of conventional PMMA, which is chemically neutral. By incorporating inorganic materials into the PMMA matrix as described herein, the pH of the resulting material is increased, thereby conferring the aforementioned advantages. The acid pH observed in PMMA also indicates incomplete polymerization of the material.

Other evaluation consists of differential scanning calorimetry (DSC), wherein samples were heated to 200° C. to quantify the residual energy required to complete polymerization. PMMA samples demonstrated reaction energies exceeding 80 J/g, whereas IIP samples exhibited reaction energies below 20 J/g, indicating that IIP provides a significantly higher degree of matrix curing. The invention therefore addresses a known deficiency associated with conventional polymeric matrices, the low polymer conversion rate.

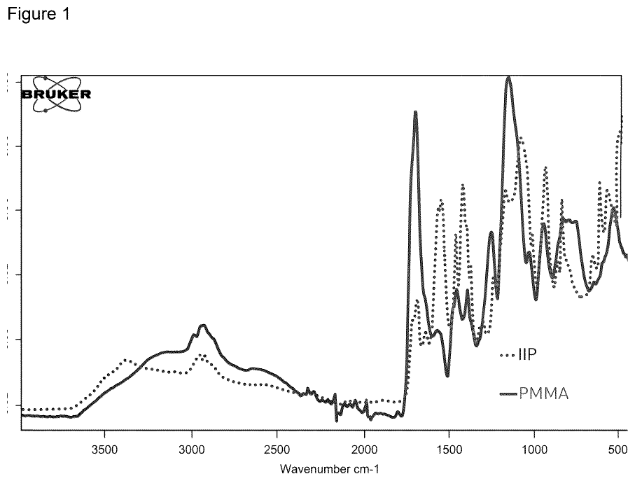

Another evaluation is Fourier-transform infrared spectroscopy (FTIR). The Fourier-transform infrared (FTIR) spectra of the materials under evaluation (FIG. 1) demonstrate clear distinctions between the material identified herein as IIP (red trace), comprising a polymethyl methacrylate (PMMA) matrix containing imprinted akermanite particulates, and neat PMMA (blue trace). Across the spectral region of 3600 to 2800 cm⁻¹, corresponding to aliphatic C—H stretching vibrations and potential surface O—H contributions, the IIP material exhibits a generalized reduction in absorbance intensity relative to neat PMMA. Such reduction is consistent with a decreased presence of free methyl and methylene groups and is therefore indicative of a higher degree of monomer conversion within the IIP matrix. In the region between 1750 and 1700 cm⁻¹, which corresponds to the ester carbonyl (C═O) stretching vibration characteristic of PMMA, both spectra exhibit the expected absorption band. However, the intensity of this band in the IIP material is comparatively diminished, suggesting a reduced quantity of residual carbonyl functionalities associated with unreacted or partially polymerized methacrylic monomer. This reduction is consistent with the enhanced curing behavior attributed to the IIP composition. Within the 1500 to 1300 cm⁻¹ region, associated with CH₂ and CH₃ deformation modes, the IIP spectrum displays variations in intensity as well as minor peak shifts when compared to neat PMMA. These spectral deviations are consistent with interactions between the PMMA matrix and ionic species released from the akermanite component, including calcium and magnesium ions, which are capable of influencing the vibrational environment of the polymeric functional groups. More pronounced differences are observed in the 1300 to 1000 cm⁻¹ region, corresponding to C—O—C and C—O stretching vibrations of the ester functionality. While PMMA exhibits well-defined absorptions in this region, the IIP material displays broadened bands, redistributed intensities, and alterations in peak shape. This spectral behavior aligns with known vibrational signatures of silicate-based ceramics, including akermanite, whose Si—O—Si and Si—O—Mg modes typically fall within the 1100 to 900 cm⁻¹ region. The modifications observed are therefore consistent with the incorporation of silicate structures into the polymer matrix. Below 900 cm⁻¹, and particularly within the 850 to 500 cm⁻¹ region, the IIP spectrum diverges substantially from that of neat PMMA. Whereas PMMA exhibits absorptions associated with out-of-plane C—H bending and skeletal C—C vibrations, the IIP material presents additional bands, increased complexity, and altered intensities. These features correspond to vibrational modes characteristic of calcium-magnesium silicates, including Si—O and Mg—O bending vibrations known to be present in akermanite. The appearance of such bands provides further evidence of the inorganic phase's structural contribution to the IIP spectrum. Collectively, the reductions in peak intensity, the observed peak shifts, the emergence of new absorptions, and the redistribution of spectral features within the IIP material relative to neat PMMA demonstrate that the polymeric matrix has undergone structural and chemical modification attributable to the presence of akermanite. These differences confirm that the IIP composition represents a molecularly modified system rather than a physical mixture, wherein the inorganic phase exerts measurable influence on the infrared vibrational profile of the polymer. FTIR analysis further demonstrates that IIP is not a mere physical mixture of materials, but instead constitutes a molecularly-imprinted system, as evidenced by peak shifts and spectral modifications relative to the PMMA reference spectrum.

One more evaluation is pycnometry, used to determine sample density. The IIP samples demonstrated a density of approximately 1.45 g/cm³, compared to approximately 1.30 g/cm³ for PMMA. This difference is attributable both to the inherently higher density of the resulting matrix and to the presence of the denser inorganic phase incorporated into the structure.

The last evaluation comprises scanning electron microscopy (SEM) coupled with energy-dispersive X-ray spectroscopy (EDS). Samples of PMMA and IIP were examined in solid form (post-synthesis) and in powdered form (post-milling).

Regarding IIP, EDS analysis reveals the elemental presence of silicon, magnesium, and calcium from akermanite, as well as the carbon and oxygen present in PMMA. This indicates a macroscopic chemical interaction between these elements. When compared to pure PMMA, only traces of carbon and oxygen are present.

Still regarding IIP, it is again possible to observe through EDS analysis at both magnifications the elemental presence of silicon, magnesium, and calcium from akermanite, as well as the carbon and oxygen present in PMMA. When compared to pure PMMA, these only show traces of carbon and oxygen (and sodium, which may be a trace of some impurity present in the sample). With 1000× magnification, it is possible to infer that there are interactions at the molecular level between the two substances, in order to characterize the imprint of inorganic particulates on the polymeric matrix.

The molecular-level interaction present in IIP could be noted where akermanite particulates are imprinted on the polymer matrix and available in the structure (without being enclosed in the polymer matrix), that is, they can act directly in ionic release. This corroborates what was discussed earlier in paragraphs 0076 and 0077, where it was possible to observe higher pH values of the aqueous dispersion of IIP compared to PMMA, precisely because of the availability of inorganic particulates for ion exchange of ions such as calcium and magnesium.

In a representative embodiment, the inorganic-imprinted polymer (IIP) comprises approximately 40%-50% by weight of akermanite and 50-60% by weight of a polymeric matrix. Considering the known stoichiometry of akermanite (Ca₂MgSi₂O₇), the inorganic phase inherently contains calcium and magnesium in proportions of approximately 29% Ca and 9% Mg by weight. Accordingly, in a 1 g sample of the IIP, the total amounts of calcium and magnesium present in the inorganic fraction correspond, respectively, to approximately 118 mg of Ca and 36 mg of Mg.

When such a material is placed in contact with an aqueous medium, only a fraction of these ions is released into solution under typical experimental or physiological conditions. Depending on the time of exposure, pH, temperature, and surface area of the micronized material, the concentration of ions measured in the aqueous extract may range from a small percentage of the theoretical maximum to several percent of the total available content. In certain embodiments, the concentration of calcium ions detectable in the aqueous phase may fall within the range of 300 to 3,000 ppm, while the concentration of magnesium ions may fall within the range of 90 to 900 ppm. These ranges reflect partial dissolution of the inorganic phase and are consistent with controlled ion-release behavior characteristic of silicate-based bioceramic systems.

Such concentrations are readily detectable through analytical methods including, but not limited to, inductively coupled plasma optical emission spectroscopy (ICP-OES), inductively coupled plasma mass spectrometry (ICP-MS), and atomic absorption spectroscopy (AAS). It is understood that the exact ionic concentrations may vary in accordance with the composition, particle size distribution, processing parameters, and environmental conditions employed in each embodiment.

1. A dental composition comprising a micronized and densified inorganic-imprinted polymer (IIP), wherein the polymer is derived from a solid mixture of functional monomers and is imprinted with inorganic particles.

2. The dental composition of claim 1, wherein the functional monomers are methacrylate monomers.

3. The dental composition of claim 2, wherein the methacrylate monomers are methacrylic acid derivatives, phosphocholine derivatives, ethoxylated monomers, or any other monomer capable of forming polymeric chains.

4. The dental composition of claim 1, wherein the inorganic particles are silicates and/or ionic salts.

5. The dental composition of claim 1, wherein inorganic particles are selected from salts, silicates, aluminates, titanates, and/or phosphosilicates each of which containing ions selected from the group consisting of Ca, Mg, Sr, and Zn.

6. The dental composition of claim 1, wherein the inorganic particles are silicates.

7. The dental composition of claim 1 further comprising a carrier and/or excipient.

8. The dental composition of claim 1, wherein the inorganic-imprinted polymer (IIP) is capable of reversible and rechargeable ion exchange with a surrounding medium under hypo-saturated and hyper-saturated ionic conditions.

9. A method for inducing tissue regeneration, the method comprising placing a dental composition into the tissue to be regenerated, wherein the dental composition comprises a micronized and densified inorganic-imprinted polymer (IIP), wherein the polymer is derived from a solid mixture of functional monomers and is imprinted with inorganic particles, such that inorganic ions are released from the placed dental composition and induce mineral deposition and mineralized tissue formation, thereby inducing the tissue regeneration.

10. The method of claim 9, wherein the functional monomers are methacrylate monomers.

11. The method of claim 10, wherein the methacrylate monomers are methacrylic acid derivatives, phosphocholine derivatives, ethoxylated monomers, or any other monomer capable of forming polymeric chains.

12. The method of claim 9, wherein the inorganic particles are silicates and/or ionic salts.

13. The method of claim 9, wherein inorganic particles are selected from salts, silicates, aluminates, titanates, and/or phosphosilicates each of which containing ions selected from the group consisting of Ca, Mg, Sr, and Zn.

14. The method of claim 9, wherein the inorganic particles are silicates.

15. The method of claim 9, wherein the dental composition further comprises a carrier and/or excipient.

16. The method of claim 9, wherein the inorganic-imprinted polymer (IIP) is capable of reversible and rechargeable ion exchange with a surrounding medium under hypo-saturated and hyper-saturated ionic conditions.
